(19) United States
(12) Patent Application Publication
(10) Pub. No.: US 2003/0125412 A1
(43) Pub. Date: Jul. 3, 2003

(54) Low shrinking polymerizable dental material

(75) Inventors: Joachim E. Klee, Radolfzell (DE); Uwe Walz, Konstanz (DE); Dirk Holter, Lorrach (DE); Armin Burgath, Bodman (DE); Holger Erey, Freiburg (DE); Rolf Mulhaupt, Freiburg (DE)

Correspondence Address: Douglas J. Hura, DENTSPLY INTERNATIONAL INC., 570 West College Avenue, York, PA, 17405-0872, US

(73) Assignee: DENTSPLY DeTrey GmbH

(21) Appl. No.: 10/321,689

(22) Filed: Dec. 17, 2002

Related U.S. Application Data
(63) Continuation of application No. 10/016,330, filed on Dec. 11, 2001, now abandoned.
Application No. 10/016,330 is a continuation of application No. 09/543,199, filed on Apr. 5, 2000, now abandoned.
Application No. 09/543,199 is a continuation of application No. 08/960,955, filed on Oct. 30, 1997, now abandoned.
(60) Provisional application No. 60/038,812, filed on Feb. 21, 1997.

(51) Int. Cl. A61F 2/00; C08K 3/40

(52) U.S. Cl. 523/114; 523/115; 524/494

(57) ABSTRACT

The invention describes a low shrinking polymerizable dental material, comprising a mixture of a polymerizable resin, a polymerizable monomer, a polymerization initiator and/or sensibilizer and a filler in a content of about 20 to about 85 percent. The volumetric shrinkage during polymerization is less than about 1.5 Vol.-% due to its rheopex behavior.

### RELATED APPLICATION

This application is a continuation of Ser. No. 09/543,199 filed Apr. 5, 2001 (Case 1904 CON) which is a continuation of Ser. No. 08/960,955, which claims the benefit of U.S. Provisional Application Serial No. 60/038,812 filed on Feb. 21, 1997.

### TECHNICAL FIELD

The present invention is directed toward a low shrinking polymerizable dental material. More particularly, the material includes a polymerizable resin and a polymerizable monomer. The material also includes a polymerization initiator and/or sensibilizer and a stabilizer and a filler component. The material has a volumetric polymerization shrinkage of less than 1.5%, and it stiffens upon application of shear stress and/or pressure and does not relax within a predetermined working time, due to its rheopex rheologic behavior.

### BACKGROUND OF THE INVENTION

Dental filling materials mainly consist of polymerizable organic monomers and/or polymers, polymerizable monomers, polymerization initiators, and fillers. Today, the main disadvantage using composites as dental filling materials is the relatively high shrinkage of organic monomers during polymerization. The shrinkage causes the well known effect of contraction gaps and subsequent cracks. Common dental composites show a volumetric shrinkage (ΔV) of as much as 2.5 up to 4.0% or more.

Special monomers such as tricyclodecane derivatives, polyols, urethane dimethacrylates of diisocyanates and hydroxyalkylmethacrylates (as disclosed for example in EP-A 0023686, DE-A 3703120, and DE-A 3703080) show a relatively low volumetric shrinkage which give reason to suppose that the use of monomers with a higher molecular weight would be successful in the application for dental composites.

Furthermore, spiroorthoesters, spiroorthocarbonates and bicycloorthoesters (W. J. Baily, J. Macromol. Sci. Chem. A9 (1975) 849, T. Endo, Macromolecules 25 (1992) 625-628) were synthesized which show only a small volumetric shrinkage or which expand during polymerization. However, most also show a volumetric shrinkage. Frequently, expansion was found when measuring the density of crystalline monomers, that their degree of polymerization is selectively low.

In order to obtain a low water absorption composite 2.2-Bis-[4-(2-hydroxy-3-meth-acryloyloxypropoxy)-phenyl]-propane was acetylated (Kyu Ho Chae, Pollimo 17 (1993) 729). Furthermore, oligo(lactone) macromonomers were prepared by reaction of 2.2-Bis-[4-(2-hydroxy-3-meth-acryloyloxypropoxy)-phenyl]-propane and dilactide (B. Sandner, Makromol. Symp. 103 (1996) 149).

Recently, new types of α, ω-methacyloyl-terminated macromonomers comprising dicarboxylic acid moieties, phenol moieties (J. E. Klee et al. Acta Polym. 44 (1993) 163, DE 4217761) or amine structural units (Acta Polym. 42 (1991) 17, Poly. Bull. 27 (1992) 511, DD 277689, DD 279 667) were described. All of them show a relatively low volumetric shrinkage of ΔV=1.2 to 2.5% but a relatively high viscosity of about η_{23° C.}=2000 Pas.

It is well known that the shrinkage directly depends on the molecular weight of polymerizable organic monomers. On the other hand, increasing molecular weights of the monomers are combined with an increasing viscosity of the resin. Therefore, polymerizable monomers, such as oligoethyleneglycol dimethacrylates, are used to obtain a lower viscosity and the possibility to incorporate the desired amount of fillers. However, polymerizable monomers show a relatively high shrinkage by themselves, for example 12.89 vol.-% for pure triethyl-eneglycol dimethacrylate. Consequently, the application of these macromonomers results in a volumetric shrinkage of about 2.5 to 4 vol.-% of a dental composite.

Recently, hyperbranched polyesters (WO 96/07688) and dendrimers for dental application (EP 0716103=Can. Pat. 2,051,333) were described.

A need exists therefore, for a dental material which will accomplish the task for which a dental material is required, but which has a lower volumetric shrinkage.

### SUMMARY OF THE INVENTION

It is therefore, an object of the invention to provide a dental material useful, for example as a filling material or the like.

It is another object of the invention to provide such a material having a respectively lower volumetric shrinkage after polymerization, as compared to those materials heretofore known in the industry.

It is a further object of the invention to provide such a material which will stiffen upon the application of shear stress and/or pressure.

It is still another object of the invention to provide such a material which can then be cured by conventional techniques such as by the use of chemical curing agents, light radiation or the like.

These and other objects of the invention which will become apparent from the discussion to follow, are carried out by the invention as hereinafter described and claimed.

In general, a low shrinking polymerizable dental material comprises a mixture of (I) at least one polymerizable resin; (ii) at least one polymerizable monomer; (iii) at least one polymerization initiator and/or sensibilizer and a stabilizer; and, (iv) at least one filler component in a content of 20 to 85 percent by weight. The material has a volumetric polymerization shrinkage of less than about 1.5%. The material stiffens upon application of shear stress and/or pressure and does not relax within a predetermined working time due to its rheopex rheologic behavior.

The polymerizable resin is for example, an epoxide-amine macromonomer, an epoxide-dicarboxylic acid macromonomer, an epoxide-diphenol macromonomer, an addition product of amines having at least two NH functions and acrylate methcrylates, a (meth)acryloyl terminated hyperbranched polyester, having at least an ethylenically unsaturated moiety, mixtures thereof and the like.

The polymerizable resin is a macromonomer or an addition product of amines having at least two NH functions and acrylate methcrylates having a molecular mass of from about 500 to about 5000 g/mol, mixtures thereof and the like. For example, the polymerizable resin can be a (meth)acryloyl terminated hyperbranched polymer having a molecular mass of from about 2000 to about 25000 g/mol.

The polymerizable monomer is for example, a mono- and polyfunctional acrylate or methacrylate, such as diethyleneglycol dimethacrylate, triethyleneglycol dimethacrylate, 3,(4),8,(9)-dimethacryloyloxymethyltricyclodecane, dioxolan bismethacrylate, vinyl-, vinylen- or vinyliden-, acrylic- or methacrylic substituted spiroorthoesters, spiroorthocarbonates or bicyloorthoesters, glycerin trimethacrylate, trimethylol propane triacrylate, furfurylmethacrylate in a content of 5 to 50 wt-% (weight percent), mixtures thereof and the like.

The polymerization initiator and/or sensibilizer is preferably, a photoinitiator, such as benzoinmethylether, benzilketal, camphor quinone, acylphos-phinoxides in a content of 0.1 to 3 wt-%, mixtures thereof and the like. For example, the polymerization initiator can be a redox initiator such as dibenzoylperoxide/aromatic or aliphatic tert. amine, tert. butyl peroxy benzoate/ascorbic acid/metal compound in a content of 0.1 to 3 wt-%, mixtures thereof and the like.

The filler includes inorganic compounds such as La₂O₃, ZrO₂, BiPO₄, CaWO₄, BaWO₄, SrF₂, Bi₂O₃, porous glasses or organic fillers, such as polymer granulate or a combination of organic and/or inorganic fillers or reactive inorganic fillers, mixtures thereof and the like. The fillers preferably have an average diameter of less than 10 μm.

### PREFERRED EMBODIMENTS FOR CARRYING OUT THE INVENTION

There is provided according to the present invention, a low shrinking polymerizable dental material. The material preferably includes a mixture of (i) at least one polymerizable resin; (ii) at least one polymerizable monomer; (iii) at least one polymerization initiator and/or sensibilizer and a stabilizer; and, (iv) at least one filler in a content of about 20 to about 85 percent by weight. By “low shrinking” it is meant a material having a a volumetric polymerization shrinkage of less than about 1.5%. The present material stiffens upon the application of shear stress and/or pressure and does not relax within a predetermined working time of the material due to its rheopex rheologic behavior (the tendency to stiffen upon being so agitated).

The polymerizable resin is preferably an epoxide-amine macromonomer, an epoxide-dicarboxylic acid macromonomer, an epoxide-diphenol macromonomer, an addition product of amines having at least two NH functions and acrylate methacrylates, a (meth)acryloyl terminated hyperbranched polyester, having at least an ethylenically unsaturated moiety, mixtures thereof and the like.

The polymerizable resin is preferably a macromonomer having a molecular mass of about 500 to about 5000 g/mol or a (meth)acryloyl terminated hyperbranched polymer having a molecular mass of about 2000 to about 25000 g/mol, mixtures thereof and the like.

Useful polymerizable monomers include mono- and polyfunctional acrylates or methacrylates, such as diethyleneglycol dimethacrylate, triethyleneglycol dimethacrylate, 3,(4),8,(9)-dimethacryloyloxymethyltricyclodecane, dioxolan bismethacrylate, vinyl-, vinylen- or vinyliden-, acrylic- or methacrylic substituted spiroorthoesters, spiroorthocarbonates or bicyloorthoesters, glycerin trimethacrylate, trimethylol propane triacrylate, furfurylmethacrylate in a content of about 5 to about 50 wt-%, mixtures thereof and the like.

The photoinitiator is preferably benzoinmethylether, benzilketal, camphor quinone/amine, or an acylphosphinoxide in a content of about 0.1 to about 3 wt-%, mixtures thereof and the like.

Useful redox initiators are dibenzoylperoxide/aromatic or aliphatic tert. amine, tert. butyl peroxy benzoate/ascorbic acid/metal compound in a content of about 0.1 to about 3 wt-%, mixtures thereof and the like.

The low shrinking dental material is preferably filled with inorganic compounds such as La₂O₃, ZrO₂, BiPO₄, CaWO₄, BaWO₄, SrF₂, Bi₂O₃, porous glasses or organic fillers, such as polymer granulate or a combination of organic and/or inorganic fillers or reactive inorganic fillers having a average diameter of less than about 10 μm, mixtures thereof and the like.

Other useful components will be exemplified hereinbelow. These materials provide a workingtime within a target range of from about 0.5 to about 3 minutes.

For example a composite was prepared using a acetylated 2,2-Bis-[p-(2-hydroxy-3-methacryloyloxypropoxy)-phenyl]-propane, a modified macromonomer M-C11 using undecanoic acid, trimethylolpropane triacrylate, champhor quinone, DMABE, BHT and a Strontium-Alumo-Fluoro-Silicate glass. Using a curing unit (Dentsply De Trey) the composite was polymerized by irradiation with visible light during 40 seconds. The obtained material shows under shear a volume shrinkage of ΔV=1.07±0.09% and a compressive strength of 238±7 MPa, a flexural strength of 68±12 MPa and a E-modules of 5786±295 MPa. Without shear or pressure the volume shrinkage is ΔV=1.98±0.12% (Archimedes method).

The volumetric shrinkage under shear stress is measured using a machine of the Zahnklinik of Zurich (Zurich machine). Using this machine the composite material is put between a glass plate and a metal plate. Then, the material is polymerized photochemically and the change of the high of the material is registered by using a photodiode or other detector.

The Archimedes method for estimation of the shrinkage is based on the measurement of the weight of the unpolymerized and of the polymerized material on air and in water. From these values the densities are calculated. The densities of the unpolymerized and of the polymerized material are used for calculating the shrinkage.

### GENERAL EXPERIMENTAL

In order to demonstrate the practice of the present invention, a number of example materials were prepared and tested. Comparisons to commercially available products were also made as will be described hereinbelow.

### Example 1

To a mixture of 37.91 g (205.82 mmol) of ethyleneglycol acrylate methacrylate (EGAMA) and 37.91 mg BHT dissolved in 100 ml of methanol were added 10.00 g (51.46 mmol) of 3,(4),8,(9)-diaminomethyltricyclodecane at 0-5° C. Than the mixture were stirred for further 2 hours at room temperature, the solvent was removed and the mixture were kept for further reaction for 24 hours at room temperature.

### Application Example 1

A composite was prepared using 8.2296 g of the polymerizable resin of Example 1, 19.3496 g of 2,2-Bis-[p-(2-hydroxy-3-methacryloyloxypropoxy)-phenyl]-propane (Bis-GMA), 0.0965 g champhor quinone, 0.0956 g N,N-dimethyl-p-amino benzoic acid ethyl ester (DMABE), 0.0278 g 2.6-di-tert.butyl-p-cresol (BHT) and 72.2000 g of a Strontium-Alumo-Fluoro-Silicate glass. Using a curing unit (Dentsply De Trey) the composite was polymerized by irradiation with visible light during 40 seconds. The obtained material shows under shear a volume shrinkage of ΔV=1.10±0.17% and a compressive strength of 221±12 MPa, a flexural strength of 52±13 MPa and a E-modules of 5955±510 MPa. Without shear or pressure the volume shrinkage is ΔV=2.89±0.08% (Archimedes method).

### Example 2

To a mixture of 37.28 g (202.40 mmol) of EGAMA and 37.31 mg BHT dissolved in 100 ml of methanol were added 7.50 g (50.60 mmol) of 3.6-dioxaoctane diamine-1.8 at 0-5° C. Than the mixture were stirred for further 2 hours at room temperature, the solvent was removed and the mixture were kept for further reaction for 24 hours at room temperature.

### Application Example 2

A composite was prepared using 8.3166 g of the polymerizable resin of example 2, 19.2629 g of Bis-GMA, 0.0965 g champhor quinone, 0.0956 g DMABE, 0.0275 BHT and 72.2000 g of a Strontium-Alumo-Fluoro-Silicate glass. Using a curing unit (Dentsply De Trey) the composite was polymerized by irradiation with visible light during 40 seconds. The obtained material shows under shear a volume shrinkage of ΔV=1.05±0.05% and a compressive strength of 224±6 MPa, a flexural strength of 61±4 MPa and a E-modules of 3847±288 MPa. Without shear and/or pressure the volume shrinkage is ΔV=2.69±0.09% (Archimedes method).

### Example 3

Synthesis of Modified 2,2-Bis-[-(2-hydroxy-3-methacrloyloxypropoxy)-phenyl]-propane (Bis-GMA-C2) 76.00 g (148.26 mmol) of Bis-GMA, 17.81 g (296.53 mmol) of acidic acid and 2.90 g of dimethylaminopyridin were dissolved in 300 ml CH₂Cl₂/DMF (5:3). To this mixture were added 67.30 g (326.18 mmol) of dicyclohexyl carbodiimid at 0° C. and stirred for 15 minutes at 0° C. and for 26 hours at room temperature. Thereafter the solid dicyclohexyl urea was filtered off. To the filtrate were added 75.8 mg of BHT and the solvent was evaporated in vacuum. Than the product was dissolved in 100 ml CH₂Cl₂ and cooled to 0° C. The filtrate was washed twice by using of 150 ml 1n HCl, 150 ml of 1n NaHCO₃-solution and 150 ml of water. Than the solution was dried over NaSO₄ and the solvent was evaporated.

Yield: 46.63 g (49.7% of th.), η_{23° C.}=86.73 Pas

Synthesis of an Epoxide-dicarboxylic Acid Macromonomer (M)

16.742g (114.60 mmol) adipic acid, 78.000 g (229.20 mmol) 2.2-bis[4-(2.3-epoxypropoxy) phenyl]-propane, 19.726 g (229.20 mmol) methacrylic acid, 1.040 g triethylbenzyl ammonium chloride and 0.106 g BHT were reacted for four hours at 90° C. The obtained methacrylate terminated macromonomer is soluble in organic solvents such as chloroform, DMF and THF. In the IR-spectrum no absorption of epoxide groups at 915 and 3050 cm⁻¹ was observed but a new absorption of ester groups was found at 1720 cm⁻¹.

| | | | |
|---|---|---|---|
| Mₙ(vpo) = 1050 g/mol, T_{g} = 13.9° C. | | | |
| (C₅₆H₇₀O₁₆), 999.17 g/mol | calc. | C 67.32 | H 7.06 |
| | found | C 67.37 | H 7.34 |

Synthesis of Modified Epoxide-dicarboxylic Acid Macromonomer (M-C11)

65.00 g (65.05 mmol) of the macromonomer (M), 48.48 g (260.22 mmol) of undecanoic acid and 2.54 g of dimethylaminopyridin were dissolved in 300 ml CH₂Cl₂/DMF (5:3). To this mixture were added 59.06 g (286.24 mmol) of dicyclohexyl carbodiimid at 0° C. and stirred for 15 minutes at 0° C. and for 26 hours at room temperature. Thereafter the solid dicyclohexyl urea was filtered off. To the filtrate were added 95.5 mg of BHT and the solvent was evaporated in vacuum. Than the product was dissolved in 100 ml CH₂Cl₂ and cooled to 0° C. The filtrate was washed twice by using of 150 ml 1n HCl, 150 ml of 1n NaHCO₃-solution and 150 ml of water. Than the solution was dried over NaSO₄ and the solvent was evaporated.

Yield: 86.90 g (79.9% of th.), η_{23° C.}=21.41 Pas

### Application Example 3

A composite was prepared using 13.0938 g of the modified Bis-GMA-C2 of example 3, 13.0938 g of the modified macromonomer M-C11 of example 3, 1.390 of trimethylolpropane triacrylate, 0.0973 g champhor quinone, 0.0973 g DMABE, 0.0278 g BHT and 72.2000 g of a Strontium-Alumo-Fluoro-Silicate glass. Using a curing unit (Dentsply De Trey) the composite was polymerized by irradiation with visible light during 40 seconds. The obtained material shows under shear a volume shrinkage of ΔV=1.07±0.09% and a compressive strength of 238±7 MPa, a flexural strength of 68±12 MPa and a E-modules of 5786±295 MPa. Without shear and/or pressure the volume shrinkage is ΔV=1.98±0.12% (Archimedes method).

When the volumetric shrinkage of the composite is measured after a time of 20, 60, 90 and 180 seconds, an unchanged shrinkage is obtained. Obviously, the relaxation from the rheopex state needs a longer time than the dental working time.

| time of storage | ΔV |
|---|---|
| s | % |
| 20 s | 1.17 ± 0.17 |
| 60 s | 1.07 ± 0.09 |
| 90 s | 0.74 ± 0.14 |
| 180 s | 1.23 ± 0.08 |

### Example 4

Synthesis of a Hyperbranched Polyester of the 2nd Generation (HHG2-OH)

134.2 g (1 mol) of 2.2-bis(methylol)propionic acid (Bis-MPA), 14.9 g (0.111 mol) of tris(methylol)propane (TMP) and 0.671 g of p-toluenesulfonic acid were mixed in a three necked flask equipped with a nitrogen inlet, a drying tube and a stirrer. Subsequently the flask was placed in a oil bath previously heated to 140° C. and the mixture was stirred at this temperature for 2 hours under a stream of nitrogen. Afterwards the nitrogen stream was turned off and the mixture dried for two hours in vacuum at 140° C., yielding HHG2-OH.

Synthesis of a Hyperbranched Polyester of the 3rd Generation (HHG3-OH)

49.17 g of HHG2-OH (corresponds to 0.5 mol OH-groups assuming complete conversion), 67.07 g of Bis-MPA and 0.335 g of p-toluenesulfonic acid were mixed in a three necked flask equipped with a nitrogen inlet, a drying tube and a stirrer. Subsequently the flask was placed in a oil bath previously heated to 140° C. and the mixture was stirred at this temperature for 2 hours under a stream of nitrogen. Afterwards the nitrogen stream was turned off and the mixture dried for two hours in vacuum at 140° C., yielding HHG3-OH.

Synthesis of a Hyperbranched Polyester of the 4th Generation (HHG4-OH)

26.81 g of HHG3-OH (corresponds to 0.25 mol OH-groups assuming complete conversion), 33.53 g of Bis-MPA and 0.168 g of p-toluenesulfonic acid were mixed in a three necked flask equipped with a nitrogen inlet, a drying tube and a stirrer. Subsequently the flask was placed in a oil bath previously heated to 140° C. and the mixture was stirred at this temperature for 2 hours under a stream of nitrogen. Afterwards the nitrogen stream was turned off and the mixture dried for two hours in vacuum at 140° C., yielding HHG4-OH.

Synthesis of a Hyperbranched Polyester of the 5th Generation (HHG5-OH)

55.16 g of HHG4-OH (corresponds to 0.494 mol OH-groups assuming complete conversion), 66.25 g of Bis-MPA and 0.331 g of p-toluenesulfonic acid were mixed in a three necked flask equipped with a nitrogen inlet, a drying tube and a stirrer. Subsequently the flask was placed in a oil bath previously heated to 140° C. and the mixture was stirred at this temperature for 2 hours under a stream of nitrogen. Afterwards the nitrogen stream was turned off and the mixture dried for two hours in vacuum at 140° C., yielding HHG5-OH.

Esterification of a Hyperbranched Polyester of the 5th Generation

20.00 g (1.83 mmol) of a hyperbranched polyester of the 5th generation HHG5-OH (Mₙ 10934.26 g/mol) and 21.32 g (210.71 mmol) of triethylamine were dissolved in 100 ml THF. Under stirring and cooling a mixture of 10.29 g (96.58 mmol) of isobutyric acid and 10.10 g (96.58 mmol) of methacrylic acid in 50 THF were added. The precipitated solid was then filtered off and washed twice with THF. Thereafter 0.0322 g BHT were added and the solvent was removed. The remaining viscous liquid was dissolved in ether and washed twice by using of a saturated NH₄Cl solution. The solution was then extracted with a 2 molar NaOH solution for four to five times and dried over Na₂SO₄. The modified hyperbranched polyester was obtained by removing the solvent and drying in vacuum.

### Application Example 4

A composite was prepared using 20.7919 g of the polymerizable resin of example 4, 4.6375 g of triethylenglycoldimethacrylate, 0.0975 g champhor quinone, 0.0976 g N,N-dimethyl-p-amino benzoic acid ethyl ester, 0.0257 g di-tert. butyl-p-hydroxy toluene and 74.3500 g of a Strontium-Alumo-Fluoro-Silicate glass. Using a curing unit (Dentsply De Trey) the composite was polymerized by irradiation with visable light during 40 seconds. The obtained material shows under shear a volume shrinkage of ΔV=0.93±0.06% and a compressive strength of 215±7 MPa, a strength of 64±5 MPa and a E-modules of 4741±238 MPa. Without shear pressure the volume shrinkage is ΔV=2.25±0.07% (Archimedes method).

| | | Polymerization | | | | |
|---|---|---|---|---|---|---|
| | Polymerization | without | | | | |
| | with pressure | pressure | | Compressive | Flexural | |
| | ΔV (Z.) | ΔV (A.) | ΔV (calc.) | strength | strength | E-modules |
| Example | % | % | % | MPa | MPa | MPa |
| 1 | 1.10 ± 0.17 | 2.89 ± 0.08 | 2.69 | 221 ± 12 | 52 ± 13 | 5955 ± 510 |
| 2 | 1.05 ± 0.05 | 2.69 ± 0.09 | 2.62 | 224 ± 6 | 61 ± 4 | 3847 ± 288 |
| 3 | 1.07 ± 0.09 | 1.98 ± 0.12 | 2.18 | 238 ± 7 | 68 ± 12 | 5786 ± 295 |
| 4 | 0.93 ± 0.06 | 2.25 ± 0.07 | 2.46 | 215 ± 7 | 64 ± 5 | 4741 ± 238 |

ΔV (Z.) - Measurement of the volumetric shrinkage at the Zurich-machine
ΔV (A.) - Measurement of the volumetric shrinkage according Archimedes
ΔV (calc.) - Shrinkage calculated from shrinkage of the resin

### Comparative Examples 1-9

In the following table are summarized the results of shrinkage measurement using the Zurich-Machine (Z.) and using the Archimedes method (A.) of commercial composites as well as their mechanical properties.

| | | | Polymerization | | | |
|---|---|---|---|---|---|---|
| | | Polymerization | without | | | |
| | | with pressure | pressure | Compressive | Flexural | |
| | | ΔV (Z.) | ΔV (A.) | strength | strength | E-modules |
| Name | Producer | % | % | MPa | MPa | MPa |
| Charisma | Kulzer | 3.16 ± 0.11 | 3.12 ± 0.38 | 394 ± 43 | 93 ± 12 | 5935 ± 142 |
| Conquest | USA | — | 4.06 ± 0.14 | 346 ± 40 | 86 ± 19 | 6719 ± 441 |
| Crystall | | | | | | |
| Durafill | Kulzer | 2.63 ± 0.17 | 2.59 ± 0.07 | 399 ± 47 | 51 ± 4 | 2100 ± 216 |
| Graft LC | GC Dental Inc. | 2.60 ± 0.15 | 2.55 | — | — | — |
| Heliomolar | Vivadent | 2.06 ± 0.08 | 2.39 ± 0.17 | 350 ± 16 | 69 ± 5 | 3910 ± 126 |
| Prisma TP.H | Dentsply | 2.78 ± 0.02 | 2.95 | — | — | — |
| Prisma TP.H | Dentsply | 2.35 ± 0.21 | 3.30 ± 0.14 | 316 ± 18 | 96 ± 7 | 7670 ± 405 |
| Tetric Ceram | Vivadent | 2.26 ± 0.06 | 3.59 ± 0.26 | 373 ± 22 | 112 ± 3 | 8260 ± 1064 |
| Z 100 | 3M | — | 2.46 ± 0.27 | 502 ± 26 | 110 ± 15 | 10901 ± 648 |
| Solitaire | Kulzer | 3.73 ± 0.25 | — | 373 ± 11 | 135 ± 7 | 5747 ± 351 |

ΔV (Z.) - Measurement of the volumetric shrinkage at the Zurich-machine
ΔV (A.) - Measurement of the volumetric shrinkage according Archimedes

### Comparative Example 10

A composite comprising 25% (w/w) of a resin composed of Bis-GMA and TGDMA (70/30), 75% (w/w) of a glass filler and BHT, camphor quinone and DEABE shows a volumetric shrinkage of 3.05% when measured using the Archimedes method. When measuring the volumetric shrinkage using the Zurich machine a shrinkage in the same range of 3.35±0.07 is found if the material was stored for one minute (without of pressure). Under pressure a shrinkage of approximately 1% is found using both methods.

| | ΔV_{Archimedes} | ΔV_{Zürich} | ΔV_{calc.} |
|---|---|---|---|
| | % | % | % |
| p = 0 | 3.05 | 3.35 ± 0.07 | 3.24 |
| p | 0.84 | 1.25 ± 0.21 | — |

The data reported herein shows that the materials according to the invention and as described above, are effective in carrying out the objects of the invention. It is evident therefore, that the objects of a low shrinking dental material are carried out by the invention as herein described. All possible aspects of the invention beyond the best mode have not been necessarily described, and the scope of the invention shall only be determined by the following claims.

What is claimed is:

1. A low shrinking polymerizable dental material, comprising a mixture of
(i) at least one polymerizable resin
(ii) at least one polymerizable monomer
(iii) at least one polymerization initiator and/or sensibilizer and a stabilizer and
(iv) at least one filler in a content of 20 to 85 percent by weight;
wherein the material has a volumetric polymerization shrinkage of less than 1.5%, such that it stiffens upon shear or pressure and does not relax within a predetermined the working time of the material due to its rheopex rheologic behavior.

2. The low shrinking composite of claim 1 wherein said polymerizable resin is selected from the group consisting of an epoxide-amine macromonomer, an epoxide-dicarboxylic acid macromonomer, an epoxide-diphenol macromonomer, an addition product of amines having at least two NH functions and acrylate methcrylates, a (meth)acryloyl terminated hyperbranched polyester, having at least an ethylenically unsaturated moiety.

3. Low shrinking composite of claim 1 wherein said polymerizable resin is is selected from the group consisting of a macromonomer or an addition product of amines having at least two NH functions and acrylate methcrylates having a molecular mass of 500 to 5000 g/mol.

4. Low shrinking composite of claim 1 wherein said polymerizable resin is is selected from the group consisting of a (meth)acryloyl terminated hyperbranched polymer having a molecular mass of 2000 to 25000 g/mol.

5. Low shrinking composite of claim 1 wherein said polymerizable monomer is selected from the group consisting of a mono- and polyfunctional acrylate or methacrylate, such as diethyleneglycol dimethacrylate, triethyleneglycol dimethacrylate, 3,(4),8,(9)-dimethacryloyloxymethyltricyclodecane, dioxolan bismethacrylate, vinyl-, vinylen- or vinyliden-, acrylic- or methacrylic substituted spiroorthoesters, spiroorthocarbonates or bicyloorthoesters, glycerin trimethacrylate, trimethylol propane triacrylate, furfurylmethacrylate in a content of 5 to 50 wt-%.

6. Low shrinking composites of claim 1 wherein the polymerization initiator or sensibilizer is photoinitiator, selected from the group consisting of benzoinmethylether, benzilketal, camphor quinone, acylphosphinoxides in a content of 0.1 to 3 wt-%, and mixtures thereof.

7. Low shrinking composite of claim 1 wherein the polymerization initiators is a redox initiator selected from the group consisting of dibenzoylperoxide/aromatic or aliphatic tert. amine, tert. butyl peroxy benzoate/ascorbic acid/metal compound in a content of 0.1 to 3 wt-%.

8. Low shrinking composite of claim 1 wherein said filler is an inorganic compound selected from the group consisting of La₂O₃, ZrO₂, BiPO₄, CaWO₄, BaWO₄, SrF₂, Bi₂O₃, porous glasses and organic fillers.

9. The low shrinking composite of claim 8 wherein said filler is selected from the group consisting of polymer granulate or a combination of organic and/or inorganic fillers or reactive inorganic fillers.

10. Low shrinking composite of claim 1 wherein said fillers have an average diameter of less than about 10 μm.
